(19) United States
(12) Patent Application Publication
(10) Pub. No.: US 2025/0261950 A1
(43) Pub. Date: Aug. 21, 2025

(54) TRANSOSSEOUS BONE PLATE FOR FIBULA FLAP AND DENTAL IMPLANT RECONSTRUCTION

(71) Applicant: INDIVIDUAL IMPLANT SOLUTIONS, INC., Woodland Hills, CA (US)

(72) Inventor: Robert G. HALE, Woodland Hills, CA (US)

(73) Assignee: INDIVIDUAL IMPLANT SOLUTIONS, INC., Woodland Hills, CA (US)

(21) Appl. No.: 18/748,828

(22) Filed: Jun. 20, 2024

Related U.S. Application Data
(60) Provisional application No. 63/556,156, filed on Feb. 21, 2024.

(51) Int. Cl. A61B 17/15; A61B 17/17; A61B 17/80

(52) U.S. Cl. CPC A61B 17/151; A61B 17/1739; A61B 17/8071

(57) ABSTRACT

A mandible reconstruction and dental implant system includes a first reconstruction template, including a body, a first cutting guide and a second cutting guide, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template; and a reconstruction plate, including a main bracket, and a first plantar plate protruding orthogonally from the main bracket, the first plantar plate including an aperture.

### CROSS-REFERENCE TO RELATED APPLICATIONS

The present application claims priority to U.S. Provisional Application No. 63/556,156, filed Feb. 21, 2024, which is incorporated herein by reference in its entirety for all purposes.

### BACKGROUND

### Field of the Disclosure

The present disclosure relates to a customized bone cutting template and reconstruction plate for mandible reconstruction using a donor bone with integrated dental implant support.

### Description of the Related Art

Some dental implants replace missing teeth with root-form analogs which include titanium devices shaped in bone screws surgically placed into dentoalveolar structures with specialized platforms protruding through the crest of the dentoalveolus to mount dental prostheses. The dental implants are screwed through the crest of the dentoalveolus after drills prepare an osteotomy of appropriate diameter and length to receive the bone screw portion of a dental implant.

The foregoing description is for the purpose of generally presenting the context of the disclosure. Work of the inventors, to the extent it is described in this background section, as well as aspects of the description which may not otherwise qualify as prior art at the time of filing, are neither expressly or impliedly admitted as prior art against the present disclosure.

### SUMMARY

The foregoing paragraphs have been provided by way of general introduction, and are not intended to limit the scope of the following claims. The described embodiments, together with further advantages, will be best understood by reference to the following detailed description taken in conjunction with the accompanying drawings.

In one embodiment, the present disclosure is related to a mandible reconstruction and dental implant system, including a first reconstruction template, including a body, a first cutting guide disposed at a first edge of the body and a second cutting guide disposed at a second edge of the body, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template; and a reconstruction plate, including a main bracket, and a first plantar plate protruding orthogonally from the main bracket, the first plantar plate including an aperture.

In one embodiment, the present disclosure is additionally related to a method of reconstructing a mandible, including obtaining a dental image of the mandible including a degraded portion for resection; attaching a reconstruction template to a donor bone, the reconstruction template including a body, a first cutting guide disposed at a first edge of the body and a second cutting guide disposed at a second edge of the body, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template; forming openings in the donor bone using the first plantar plate slot template, the first bone fastener template, and the first implant template; cutting the donor bone along the first cutting guide and the second cutting guide to generate a first bone segment; attaching a reconstruction plate to the first bone segment via the opening corresponding to the first plantar plate slot template, the reconstruction plate including a main bracket and a first plantar plate protruding orthogonally from the main bracket, the first plantar plate including an aperture; attaching the reconstruction plate with the attached first bone segment to the mandible; and attaching a dental implant to the first plantar plate through the attached first bone segment via the opening formed corresponding to the implant template.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a schematic of a reconstruction template, according to an embodiment of the present disclosure.

FIG. 2 shows a schematic of the reconstruction template attached to a bone during a clinical procedure, according to an embodiment of the present disclosure.

FIG. 3A shows a schematic of the reconstruction template attached to the fibula with a view along the top, according to an embodiment of the present disclosure.

FIG. 3B shows a schematic of bone segments formed using the reconstruction template 900, according to an embodiment of the present disclosure.

FIG. 3C shows a schematic of a reconstruction plate (also known as a bone plate), according to an embodiment of the present disclosure.

FIG. 3D shows a schematic of a transalveolar dental implant (TDI) system including a dental post with a threaded apex engaged therein, according to an embodiment of the present disclosure.

FIG. 4A shows the plantar plate inserted into the plantar plate slot and secured using a positioning pin, according to an embodiment of the present disclosure.

FIG. 4B shows a schematic of the positioning pin secured to the plantar plate, according to an embodiment of the present disclosure.

FIG. 4C shows a schematic of the first bone segment attached to the reconstruction plate 999, according to an embodiment of the present disclosure.

FIG. 4D shows a schematic of a dental implant installed in a bone fragment, according to an embodiment of the present disclosure.

FIG. 4E shows a schematic of the bone template 900 with removable pieces, according to an embodiment of the present disclosure.

FIG. 5 shows a schematic of a reconstructed mandible, according to an embodiment of the present disclosure.

FIG. 6A shows a cross-sectional schematic of plane A, according to an embodiment of the present disclosure.

FIG. 6B shows a cross-sectional schematic of plane B, according to an embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

A mandibulectomy is a surgery to remove all or part of a jaw (mandible), which can be performed to remove a tumor, lesion, and/or necrosis that affects the jaw. Notably, the removed portions of the jaw can be reconstructed or replaced using bone from another part of the body, or a donor site. For example, the donor site can be the fibula bone, along with surrounding muscle, soft tissue, skin, veins, and arteries, which is also known as the fibula free flap.

During the course of the procedure to remove a portion of the mandible and reconstruct the mandible, the fibula bone can be cut into multiple segments and adjusted to fit in the removed portion of the mandible, and subsequently attached to a support structure that is fixated to the remaining mandible stumps flanking the removed portion. However, the process of aligning, stabilizing, and attaching the fibula bone segments to the support structure can often be cumbersome, inconsistent, and time consuming.

Furthermore, generally after the reconstruction of the mandible, patients can opt for another procedure to place dental implants along the reconstructed portion. This can require additional invasive work and another recovery period for the patient. This can also come at great expense to the patient since often times only the mandible reconstruction may be covered by health insurance, while the implant procedure would come at a cost to the patient.

Therefore, described herein is an apparatus, system, and method for a custom transosseous reconstruction bone plate for fibula free flap and dental implant reconstruction following a mandible resection.

In an embodiment, the bone plate can be contoured to align and fixate fibula bone segments to reconstruct a mandibular defect. The bone plate can be formed via, for example, additive manufacturing using a material such as titanium. The bone plate can include plantar plate features approximately 8 mm×8 mm in width and length and 2 mm thick, and can project horizontally from an inner surface of the bone plate and insert into osteotomy slots precisely formed into the fibula bone segments to assist in bone segment alignment. An aperture, such as a threaded 2.5 mm diameter aperture, can be included in the plantar plate and oriented to align with a planned dental implant osteotomy from the crest of the fibula used to reconstruct the fibula-mandible with dental implants to support a dental prosthesis for immediate return-to-function.

A drill guidance apparatus can be used to align and drill the dental implant osteotomy. After osteotomy, a dental post with a threaded apex feature can screw into the fibula-mandible and lock into the plantar plate feature of the bone plate. The dental implants can be inserted and locked into the plantar plate via the aperture and further fixate the fibula segments and provide a dental implant platform for immediate-function dental prosthesis reconstruction.

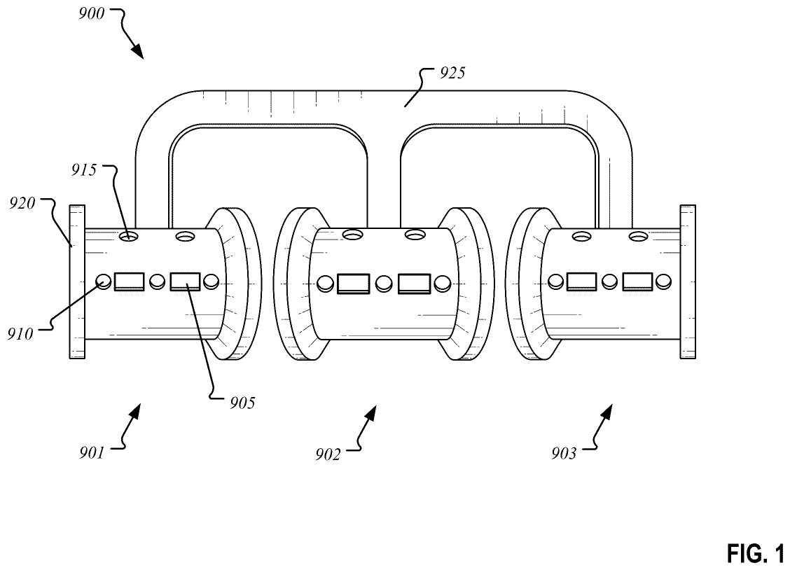

To this end, FIG. 1 shows a schematic of a reconstruction template 900, according to an embodiment of the present disclosure. In an embodiment, the reconstruction template 900 includes a first template segment 901, a second template segment 902, and a third template segment 903, but can also include any number of the template segments, such as just the first template segment 901. When the reconstruction template 900 includes more than one of the template segments, the template segments can be connected by a template segment connector 925. As shown, the template segment connector 925 can connect the first template segment 901, the second template segment 902, and the third template segment 903 together. The first template segment 901, the second template segment 902, and the third template segment 903 can each be configured to reversibly attach to a portion of a bone, such as a fibula bone, and provide aids for guiding the cutting, drilling, and adjustment of the bone to which the reconstruction template 900 is attached. For example, the first template segment 901 can include a plantar plate slot template 905, a bone fastener template 910, an implant template 915, and a cutting guide 920. In an embodiment, the first template segment 901, the second template segment 902, and the third template segment 903 can each be generally cylindrical in shape and hollow to receive the bone. As shown, the first template segment 901, the second template segment 902, and the third template segment 903 can include a shaft-like or cylindrical body with flared edges (described below). Of course, based on medical images obtained of the target bone on which the reconstruction template 900 will be attached, a shape of the first template segment 901, the second template segment 902, and the third template segment 903 can be customized to better fit on the target bone in the medical image.

In an embodiment, a material of the reconstruction template 900 is reversibly deformable (elastic) and the shape of the first template segment 901, the second template segment 902, and the third template segment 903 can be C-shaped to allow the C-shape to deform or open when forced onto the bone, and then deform back or close onto the bone to secure the reconstruction template 900 to the bone. The shape as described can be a cross-sectional shape taken orthogonal to the axis of the bone (or hollow portion of the reconstruction template 900 when a bone is not disposed therein). In an embodiment, the material of the reconstruction template 900 is rigid and the first template segment 901, the second template segment 902, and the third template segment 903 can comprise multiple portions each that can be opened, arranged on the bone, and then closed and reversibly secured in the closed state until the bone has been successfully cut, drilled, or otherwise adjusted. For example, each of the first template segment 901, the second template segment 902, and the third template segment 903 can comprise two halves (a split C-shape, or two hemispheres for a fully circular enclosed shape) that are secured to one another via a push fit lock, twist lock, fastener, clamp, etc.

For example, each of the first template segment 901, the second template segment 902, and the third template segment 903 can comprise two halves and a first half of the two halves can include a first magnet configured to attract a second complementary magnet in a second half of the two halves to reversibly couple the first half to the second half. The first magnet and the second complementary magnet can have sufficient magnetic strength when coupled to keep the first template segment 901 (or any other template segment) closed around any bone segment arranged therein during a procedure where the bone segment is relocated. In an embodiment, the first magnet and/or the second complementary magnet can be electromagnets and configured to generate a magnetic field in response to a current. In such an example, the reconstruction template 900 can include a power source, circuitry, and a switch (not shown) for each template segment to toggle the electromagnet between an on or an off state to couple or uncouple the two halves by adjusting the flow of current to the electromagnets.

In an embodiment, the shape of the first template segment 901, the second template segment 902, and the third template segment 903 can be a half-circle or less and the reconstruction template 900 can be held in place by the operator against the bone.

In an embodiment, the plantar plate slot template 905 can be an opening in the first template segment 901. The shape of the opening can have a cross-sectional shape that can be designed to receive a corresponding feature having a similar or complementary cross-sectional shape. As shown, the cross-sectional shape of the plantar plate slot template 905 can be rectangular and the plantar plate slot template 905 can be configured to receive a corresponding feature having a complementary rectangular cross-sectional shape inserted therein. For example, as will be described herein, the plantar plate slot template 905 can be configured to receive a plantar plate. The cross-sectional shape can be selected to ensure a particular orientation of the corresponding feature inserted therein, such a unique shape or shape having no rotational symmetry. Thus, the plantar plate slot template 905 can be configured to, when attached to a bone, receive an instrument or tool therein to cut, drill or otherwise adjust the bone via the opening that is the plantar plate slot template 905. While the first template segment 901 is shown as having two of the plantar plate slot template 905, the first template segment 901 can have any number n of the plantar plate slot template 905, such as only one of the plantar plate slot template 905, three of the plantar plate slot template 905, etc.

In an embodiment, the bone fastener template 910 can be an opening in the first template segment 901. The shape of the opening can have a cross-sectional shape that can be designed to receive a corresponding feature having a similar or complementary cross-sectional shape. As shown, the cross-sectional shape of the bone fastener template 910 can be circular and the bone fastener template 910 can be configured to receive a corresponding feature having a complementary circular cross-sectional shape inserted therein. For example, as will be described herein, the bone fastener template 910 can be configured to receive a fastener, such as a bone screw. Thus, the bone fastener template 910 can be configured to, when attached to a bone, receive an instrument or tool therein to cut, drill or otherwise adjust the bone via the opening that is the bone fastener template 910. For example, the bone fastener template 910 can receive a drill bit and the drill bit can remove material from the bone to provide a pilot hole for the bone screw. While the first template segment 901 is shown as having two of the bone fastener template 910, the first template segment 901 can have any number n of the bone fastener template 910, such as only one of the bone fastener template 910, three of the bone fastener template 910, etc. As shown, an arrangement of the plantar plate slot template 905 and the bone fastener template 910 on the first template segment 901 can be relatively proximal along a same line. With relation to an axis through a length of the bone, the plantar plate slot template 905 and the bone fastener template 910 can be aligned at a same rotational angle. It may be appreciated that the arrangement of the plantar plate slot template 905 and the bone fastener template 910 need not be in alignment and can be at different rotational angles to one another.

In an embodiment, the implant template 915 can be an opening in the first template segment 901. The shape of the opening can have a cross-sectional shape that can be designed to receive a corresponding feature having a similar or complementary cross-sectional shape. As shown, the cross-sectional shape of the implant template 915 can be circular and the implant template 915 can be configured to receive a corresponding feature having a complementary circular cross-sectional shape inserted therein. For example, as will be described herein, the implant template 915 can be configured to receive a dental implant post. Thus, the implant template 915 can be configured to, when attached to a bone, receive an instrument or tool therein to cut, drill or otherwise adjust the bone via the opening that is the implant template 915. For example, the implant template 915 can receive a drill bit and the drill bit can remove material from the bone to provide a guide hole to the plantar plate slot template 905 for the dental implant post to be inserted and couple with the plantar plate. While the first template segment 901 is shown as having two of the implant template 915, the first template segment 901 can have any number n of the implant template 915, such as only one of the implant template 915, three of the implant template 915, etc. As shown, an arrangement of the implant template 915 can be at an orthogonal rotational angle to the plantar plate slot template 905. That is, with relation to the axis through the length of the bone, the bone fastener template 910 and the implant template 915 can be arranged at an orthogonal rotational angle. It may be appreciated that the arrangement of the plantar plate slot template 905 and the implant template 915 need not be in any particular alignment or orthogonality and can be at different rotational angles to one another.

In an embodiment, although the plantar plate slot template 905, the bone fastener template 910, and the implant template 915 are shown as being openings having a thickness equal to a thickness of the shaft forming the main body of the first template segment 901, the second template segment 902, and the third template segment 903, the plantar plate slot template 905, the bone fastener template 910, and the implant template 915 can be raised or extruded features (and hollowed through both ends of the openings) with a substantially larger thickness in order to further help guide the drill bit inserted therein. This can ensure a desired drill angle relative to the arrangement on the shaft of the first template segment 901, the second template segment 902, and the third template segment 903. For example, an axis through the extruded openings can be orthogonal to the axis through the bone and orthogonal to a plane tangent to the location of the opening (the plantar plate slot template 905, the bone fastener template 910, or the implant template 915).

In an embodiment, the cutting guide 920 can be a flared edge of the first template segment 901 having a planar face. The planar face can be configured to aid in guiding an instrument or tool in cutting the bone. For example, a blade can press against the cutting guide 920 on one side of the bone to which the reconstruction template 900 is attached, then translated to cut through the bone while being pressed against the cutting guide 920, and the resulting cut of the bone will be a planar face as well. As shown, the cutting guide 920 along a left edge of the first template segment 901 can be orthogonal the axis of the bone (or the axis of the cylindrical shape of the first template segment 901), while the cutting guide 920 along a right edge of the first template segment 901 can have a predetermined angle. Thus, the resulting planes of the edges of the cut bone need not be parallel to one another. This can help with final fit of the cut bone segments when arranged together in the mandible to form any desired curves. In an embodiment, the angle can be customized based on a medical image obtained of the mandible. When multiple pieces of bone are needed to reconstruct the mandible, multiple of the angled cutting guide 920 can be customized for each of the template segments, such as for forming a curved mandible portion.

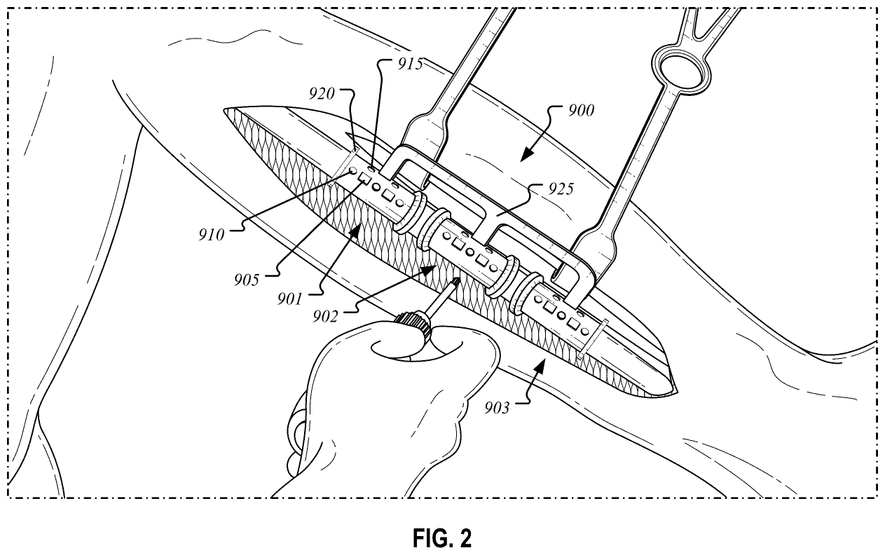

FIG. 2 shows a schematic of the reconstruction template 900 attached to a bone during a clinical procedure, according to an embodiment of the present disclosure. In an embodiment, a fibula bone (or for simplicity, also referred to as “the fibula”) of a patient can be used as the donor site for the bone needed to reconstruct the patient's mandible. Thus, as shown, the leg of the patient can be cut open to reveal the fibula, and the reconstruction template 900 can be applied or attached to the fibula.

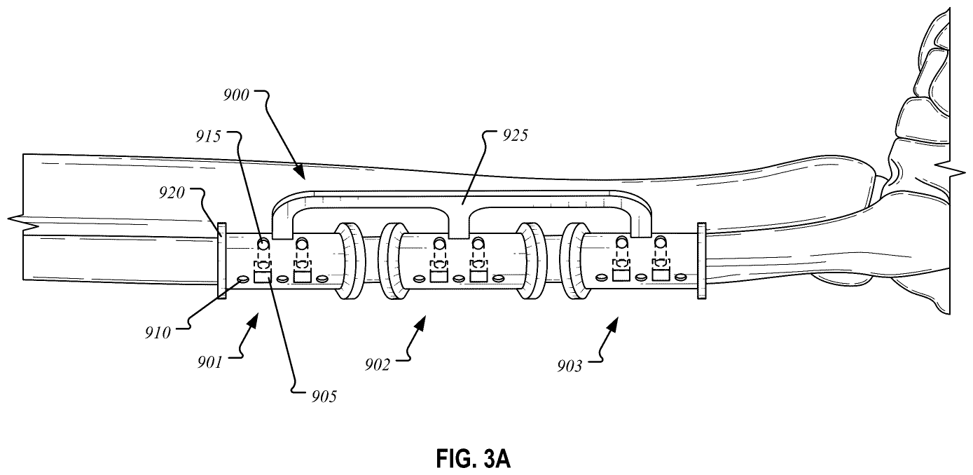

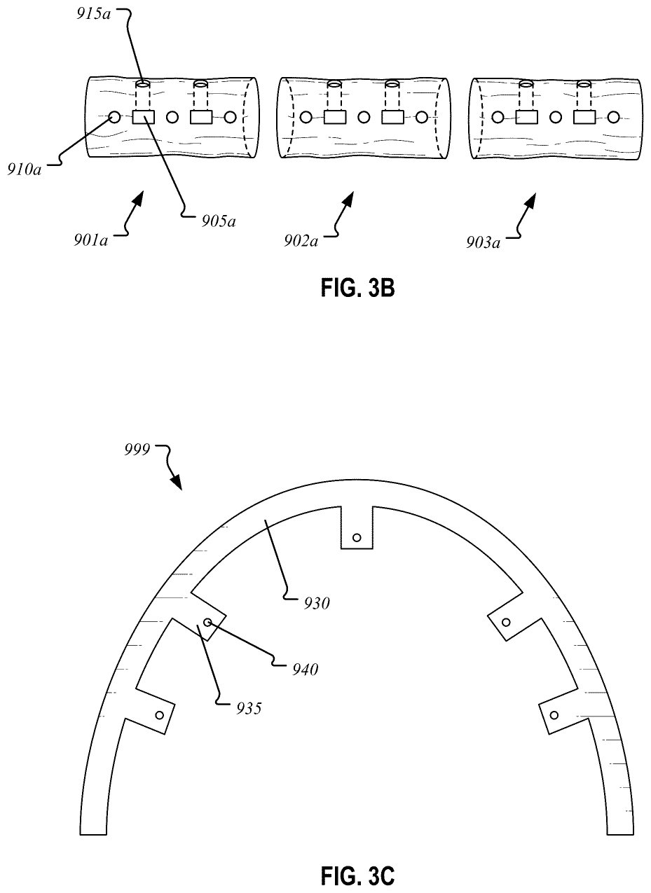

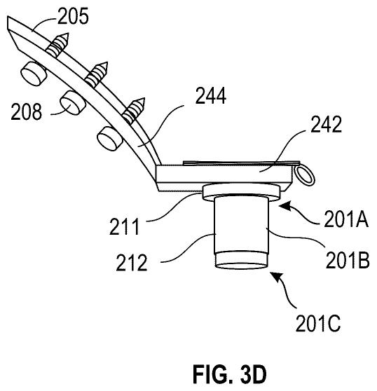

FIG. 3A shows a schematic of the reconstruction template 900 attached to the fibula with a view along the top, according to an embodiment of the present disclosure. In an embodiment, the fibula bone can be cut via the reconstruction template 900 for each of the template segments and each of the plantar plate slot template 905, the bone fastener template 910, and the implant template 915 can be used to drill or adjust the corresponding bone segments. Here, a see-through view of the implant template 915 is shown to illustrate the connection from the implant template 915 to the plantar plate slot template 905 when the bone has been drilled, where eventually a plantar plate can be inserted into the bone segment via the plantar plate slot template 905 and a dental implant can be inserted via the implant template 915 and couple with the plantar plate.

FIG. 3B shows a schematic of bone segments formed using the reconstruction template 900, according to an embodiment of the present disclosure. In an embodiment, the reconstruction template 900 can be used to generate a first bone segment 901a, a second bone segment 902a, and a third bone segment 903a. Each of the first bone segment 901a, the second bone segment 902a, and the third bone segment 903a can include a plantar plate slot 905a, a bone attachment opening 910a, and a dental implant opening 915a. As shown, the two of the plantar plate slot template 905 can be used to form the corresponding two of the plantar plate slot 905a, the three of the bone fastener template 910 can be used to form the corresponding three of the bone attachment opening 910a, and the two of the implant template 915 can be used to form the corresponding two of the dental implant opening 915a. The first bone segment 901a is also shown to have an angled cut along a right side of the first bone segment 901a, the second bone segment 902a is shown to have an angled cut along both a left and a right side of the second bone segment 902a, and the third bone segment 903a is shown to have an angled cut along a left side of the third bone segment 903a based on the angled cutting guide 920 for the reconstruction template 900 shown in FIGS. 1 and 2. Again, the dental implant opening 915a formed via the implant template 915 is shown as traversing through the bone segments to meet with the plantar plate slot 905a, and the plantar plate slot 905a can be configured to receive a plantar plate.

To this end, FIG. 3C shows a schematic of a reconstruction plate 999 (also known as a bone plate), according to an embodiment of the present disclosure. In an embodiment, the reconstruction plate 999 can be a support structure configured to couple with the first bone segment 901a, the second bone segment 902a, and the third bone segment 903a. The reconstruction plate 999 can have a shape that is based on the removed portion of the mandible. As shown in FIG. 3C, the reconstruction plate 999 can include a main bracket 930, and the main bracket 930 can be curved. The reconstruction plate 999 can be formed by, for example, additive manufacturing, molding, stamping, and CNC machining, among others. The reconstruction plate 999 can include a plantar plate 935, or multiple of the plantar plate 935, and the plantar plate 935 can include a aperture 940. The plantar plate 935 can be a protrusion from the main bracket 930 and have a rectangular shape, or any complementary shape to insert into the plantar plate slot 905a. The plantar plate 935 can be in singles or multiples to couple with the bone fragments. For the reconstruction plate 999 shown in FIG. 3C, a group of two of the plantar plate 935 can couple with a bone fragment having two of the plantar plate slot 905a, while a single of the plantar plate 935 can couple with a bone fragment having just one of the plantar plate slot 905a. A length of the plantar plate 935 can be, for example, 2 mm to 20 mm, or 4 to 15 mm, or 6 to 10 mm, or approximately 8 mm. A width of the plantar plate 935 can be, for example, 2 mm to 20 mm, or 4 to 15 mm, or 6 to 10 mm, or approximately 8 mm. A thickness of the plantar plate 935 can be, for example, 0.5 mm to 5 mm, or 1 mm to 4 mm, or approximately 2 mm. A diameter of the aperture 940 can be, for example, 1 mm to 5 mm, or 2 mm to 4 mm, or approximately 2.5 mm.

FIG. 3D shows a schematic of a transalveolar dental implant (TDI) system including a dental post with a threaded apex engaged therein, according to an embodiment of the present disclosure. In an embodiment, the TDI system can include a TDI dental post 212 with a threaded apex to frictionally lock into a corresponding threaded aperture of a previously positioned and affixed TDI bone plate 205, in situ. In an embodiment, the TDI bone plate 205 can include two portions: a first portion 242 can be a planar portion 242 and a second portion 244 can be a contoured portion 244. Notably, the planar portion 242 can be similar to the plantar plate 935, including the aperture being akin to the aperture 940 of the plantar plate 935. Similarly, the TDI dental post 212 that is installed in the aperture of the planar portion 242 can be installed in the aperture 940.

In a clinical setting, the coupling of the bone fragment to the reconstruction plate 999 via the plantar plate 935 can be easier to accomplish compared to coupling of the bone fragment to a reconstruction plate lacking the plantar plate 935. That is, once the bone fragment has been cut from the donor bone, such as the first bone segment 901a having two of the plantar plate slot 905a, the first bone segment 901a can be easily coupled with the reconstruction plate 999 by inserting the plantar plate 935 into the plantar plate slot 905a. Once coupled, the first bone segment 901a range of motion can be reduced significantly, thereby preventing the slipping of the first bone segment 901a relative to the reconstruction plate 999. As previously mentioned, this part of the reconstruction process represents one of the most clumsy, inconsistent, and time-consuming steps since operators will often try to stabilize the bone fragment by hand while trying to attach the bone fragment to the reconstruction plate since a right angle of the fastener into the reconstruction plate is desired. Furthermore, the mishandling of the bone fragments is magnified by the fluids surrounding the donor site and on the bone fragments, such as blood. Thus, the insertion of the plantar plate 935 into the plantar plate slot 905a restricts all but one range of motion, which can be further restricted by applying a temporary fastener.

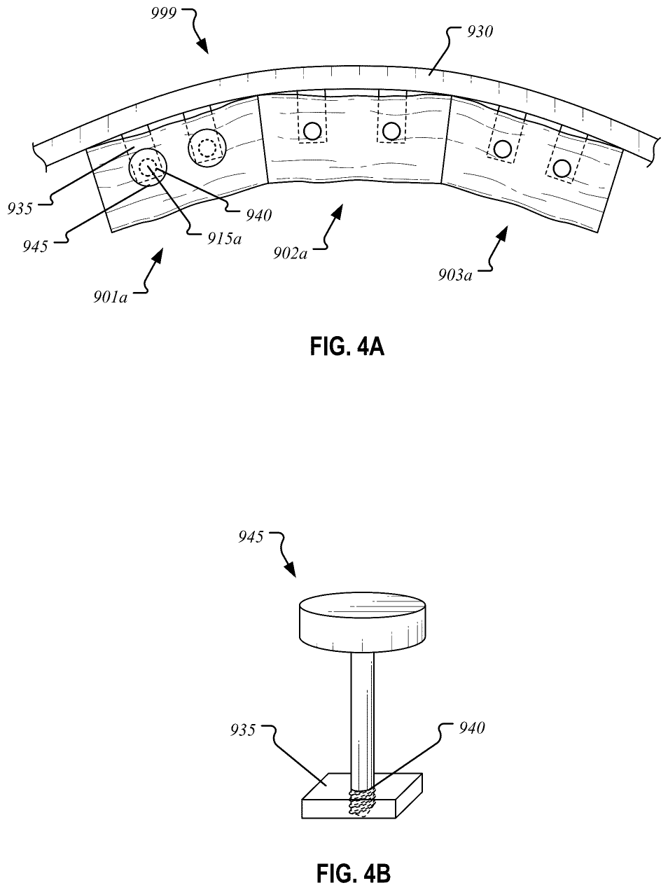

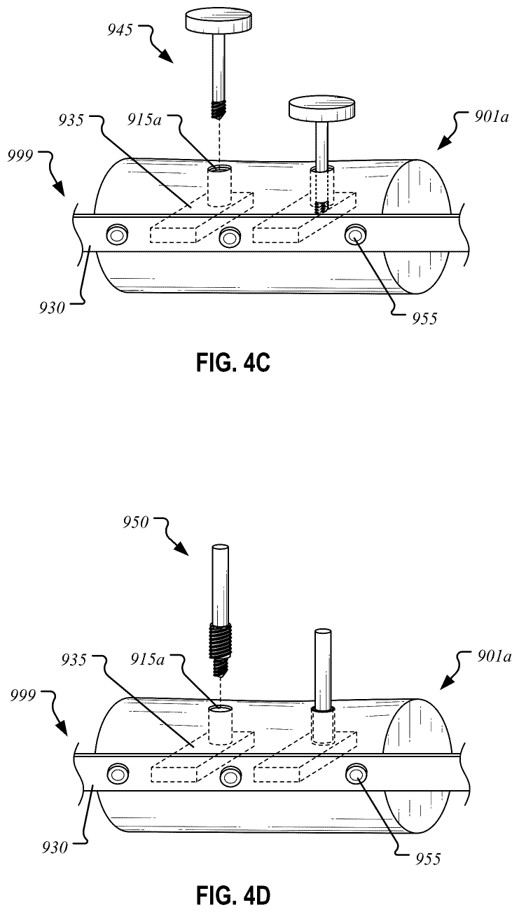

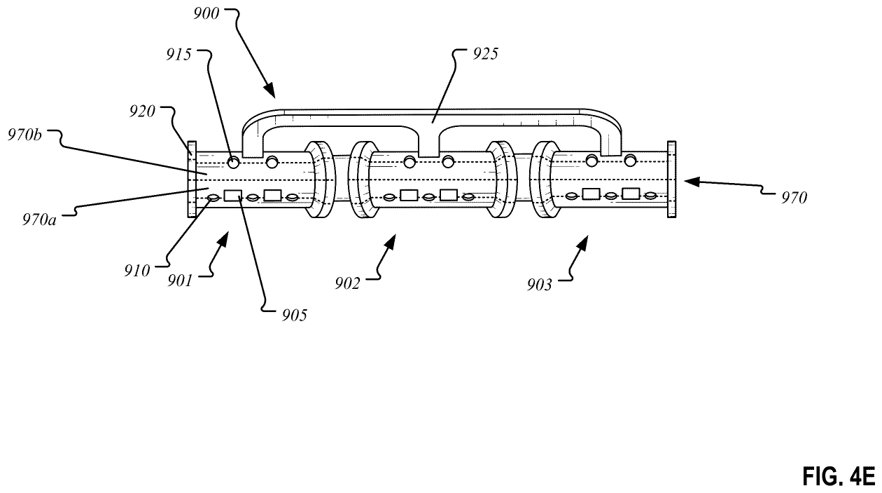

To this end, FIG. 4A shows the plantar plate 935 inserted into the plantar plate slot 905a and secured using a positioning pin 945, according to an embodiment of the present disclosure. In an embodiment, a shaft of the positioning pin 945 can be inserted into and through the dental implant opening 915a, and a tip of the positioning pin 945 can couple with the aperture 940 of the plantar plate 935.

FIG. 4B shows a schematic of the positioning pin 945 secured to the plantar plate 935, according to an embodiment of the present disclosure. In an embodiment, upon coupling of the positioning pin 945 with the plantar plate 935, the first bone segment 901a can be secured to the reconstruction plate 999 and all ranges of motion of the first bone segment 901a can be restricted. For example, the tip of the positioning pin 945 can be threaded and the aperture 940 can be threaded with a complementary threading. For example, the tip of the positioning pin 945 can be push fit into the aperture 940 having a size, shape, or dimension configured to secure the tip of the positioning pin 945 in the aperture 940. For example, the tip of the positioning pin 945 can be twist fit into the aperture 940 having a size, shape, or dimension configured to secure the tip of the positioning pin 945 in the aperture 940. For example, the tip of the positioning pin 945 can be generally friction fit into the aperture 940. For example, the tip of the positioning pin 945 can be magnetic and the aperture 940 can include a complementary magnet configured to attract the magnetic tip of the positioning pin 945.

FIG. 4C shows a schematic of the first bone segment 901a attached to the reconstruction plate 999, according to an embodiment of the present disclosure. In an embodiment, the first bone segment 901a (or the second bone segment 902a, or the third bone segment 903a) can be coupled to the reconstruction plate 999 as previously described via inserting the plantar plate 935 into the plantar plate slot 905a, and the first bone segment 901a can be temporarily secured to the reconstruction plate 999 via the positioning pin 945 inserted through the dental implant opening 915a and into the aperture 940 of the plantar plate 935. Once temporarily secured to the reconstruction plate 999, a fastener 955 (or multiple of the fastener 955) can be used to secure the first bone segment 901a to the reconstruction plate 999 via the bone attachment opening 910a. The fastener 955 can be, for example, a bone screw. As shown, the main bracket 930 of the reconstruction plate 999 can include various openings or fastening locations through which the fastener 955 can be inserted and then engaged with the first bone segment 901a. At this point in the procedure, the reconstruction plate 999 can be fixated to the flanking mandible stumps and the first bone segment 901a (and any additional bone fragments) can be coupled to the reconstruction plate 999, and the procedure can optionally end with the positioning pin 945 in place, or removed with the dental implant opening 915a covered or filled using an alternative feature. Additionally or alternatively, the procedure can continue with a dental implant implanted into the dental implant opening 915a.

To this end, FIG. 4D shows a schematic of a dental implant installed in a bone fragment, according to an embodiment of the present disclosure. In an embodiment, the mandible reconstruction can further include implanting a dental implant 950 in the first bone segment 901a. As previously described, after the reconstruction of the mandible and full recovery, patients can opt for another follow-up procedure to place dental implants along the reconstructed portion. This can require additional invasive work and another separate recovery period for the patient. This can also come at great expense to the patient since often times only the mandible reconstruction may be covered by health insurance. Thus, the implant template 915 can be used to form the dental implant opening 915a through which the dental implant 950 can be inserted and coupled to the plantar plate 935 via the aperture 940 in the same procedure. As shown, an apex of the dental implant 950 can include a fastening feature. For example, the apex of the dental implant 950 can be threaded and the aperture 940 can be threaded with a complementary threading. For example, the apex of the dental implant 950 can be push fit into the aperture 940 having a size, shape, or dimension configured to secure the apex of the dental implant 950 in the aperture 940. For example, the apex of the dental implant 950 can be twist fit into the aperture 940 having a size, shape, or dimension configured to secure the apex of the dental implant 950 in the aperture 940. For example, the apex of the dental implant 950 can be generally friction fit into the aperture 940. For example, the apex of the dental implant 950 can be magnetic and the aperture 940 can include a complementary magnet configured to attract the magnetic apex of the dental implant 950. The opposite end of the dental implant 950 from the apex can be configured to receive additional dental devices and prostheses, such as a replacement tooth. The dental implant 950 need not receive the additional dental devices and prostheses immediately and can be quickly attached to the dental implant 950 at a later point in time (without requiring an additional invasive procedure).

In an embodiment, the bone fragment(s) can remain in the bone template 900 for even easier handling during coupling of the bone segments to the bone plate. To this end, FIG. 4E shows a schematic of the bone template 900 with removable pieces, according to an embodiment of the present disclosure. In an embodiment, the bone template 900 can include, for each of the template segments, one or more breakaway portion 970 denoted by the dotted lines through the template segments. For example, as shown, the first template segment 901 includes a first breakaway portion 970a. The one or more breakaway portion 970 can be a segment of material formed as part of the first template segment 901 that is configured to separate from the first template segment 901. In such an example, the first bone fragment 901a, while still arranged in the first template segment 901, can have the plantar plate 935 inserted into the plantar plate slot 905a through the plantar plate slot template 905 of the first template segment 901. For example, the operator can hold the template segment connector with one hand and, with the other hand, manipulate the first template segment 901 (with the first bone segment 901a arranged therein) to insert the plantar plate 935 through both the plantar plate slot template 905a and the plantar plate slot 905. Upon sufficient insertion of the plantar plate 935 into the plantar plate slot 905a to prevent unwanted motion of the first bone segment 901a, the first breakaway portion 970a can be separated and removed from the first template segment 901. For example, the first breakaway portion 970a can be attached to the first template segment 901 at a plurality of connectors configured to be easily broken during the procedure to separate the first breakaway portion 970a from the rest of the first template segment 901. For example, the operator can use a small drill or saw to cut through the plurality of connectors. A material of the bone template 900 can be a polymer or metal. The bone plate 900 can be fabricated via, for example, additive manufacturing (e.g., 3D printing), injection molding, CNC machining, die casting, or similar.

As shown, for example, a first edge of the first breakaway portion 970a can run through a middle of the plantar plate slot templates 905a. Therefore, removing the first breakaway portion 970a allows the first template segment 901 to release or separate from the plantar plates 935 and the bone plate 999. Notably, when the bone fastener templates 910 are aligned with the plantar plate slot templates 905a, the fasteners 955 (described below) can also be partially fastened at this stage through the bone fastener templates 910 to further prevent unwanted motion of the first bone segment 901a and begin coupling of the first bone segment 901a to the bone plate 999. Again, since the first edge of the first breakaway portion 970a runs through a middle of the bone fastener templates 910, removing the first breakaway portion 970a allows the first template segment 901 to release or separate from the fasteners 955 and the bone plate 999. Once the first breakaway portion 970a is removed, the plantar plates 935 can be fully inserted into the plantar plate slots 905a. At this point, the fasteners 955 can be fully fastened to the first bone segment 901a as well.

When the fasteners 955 are not yet used to fasten the first bone segment 901a to the bone plate 999, the positioning pins 945 can be used instead to temporarily couple the first bone segment 901a to the plantar plate 935. The positioning pins 945 can be inserted through the implant template 915 and into the dental implant opening 915a. The first template segment 901 can include a second breakaway portion 970b with a first edge of the second breakaway portion 970b running through a middle of the implant templates 915. Therefore, removing the second breakaway portion 970b allows the first template segment 901 to release or separate from the positioning pins 945 while allowing the positioning pins 945 to remain coupled to the plantar plate 935.

As previously described, the template segments can comprise two halves (a split C-shape, or two hemispheres for a fully circular enclosed shape) that are secured to one another via, for example, a push fit lock, twist lock, fastener, clamp, magnetic force, etc. Thus, in a similar manner as described above, the first bone fragment 901a, while still arranged in the first template segment 901, can have the plantar plate 935 inserted into the plantar plate slot 905a through the plantar plate slot template 905 of the first template segment 901. Then, the two halves can be separated from one another, the first template segment 901 can be released from the plantar plate 935 (and the bone plate 999), and the plantar plate 935 can be fully inserted into the plantar plate slot 905a. For example, a push fit lock can be pulled with sufficient force to unlock the push fit lock and separate the two halves. For example, the switch can be activated to toggle power to the electromagnets securing the two halves to one another to the off state, thereby reducing the attraction of the electromagnets. Again, a set of abutting edges of the two halves can run through the middle of the plantar plate slot template 905 of the first template segment 901. Therefore, separating the two halves of the first template segment 901 allows the first template segment 901 to release or separate from the plantar plates 935 and the bone plate 999.

To summarize, an additional clinical example is described herein. In an embodiment, the fibula can be surrounded by vascular periosteum. Surgery to segmentalize the fibula can use a lateral approach with care to keep as much periosteum attached to the bone as possible. No more than, for example, a target 50% circumferential area of periosteum can be dissected off the bone. The vessels from the muscle can stay attached to the periosteum on the inner aspect of the fibula. The cutting guide 920 (C-clamps) of the bone template 900 can overlay the periosteum partially on an inner aspect in order to achieve, for example, at least 60% of clamp arranged or coupled onto the fibula.

The bone template 900, and in particular, the target template segment can be stabilized by screws through the bone fastener templates 910. The fibula can be segmentalized and the intersegment pieces (the bone segments 901a, 902a, 903a) can be removed. The intersegments can then be inserted onto plantar projections (the plantar plates 935) of the bone plate 999 and the positioning pins 945 can be arranged or inserted through the dental implant opening 915a to lock into the (plantar) apertures 940. As this is being performed, portions of the bone template 900 (the one or more breakaway portion 970) can be peeled away or removed, especially to expose the bone attachment opening 910a and the plantar plate slot 905a areas which will be seated against the main bracket 930 of the bone plate 999.

The template segment connector 925 of the bone template 900 can be removed as an early step to bring butt ends of the intersegment pieces together. The other aspects of the bone template 900 can be removed early or late depending on clinical needs to hold onto wet and slippery intersegment pieces.

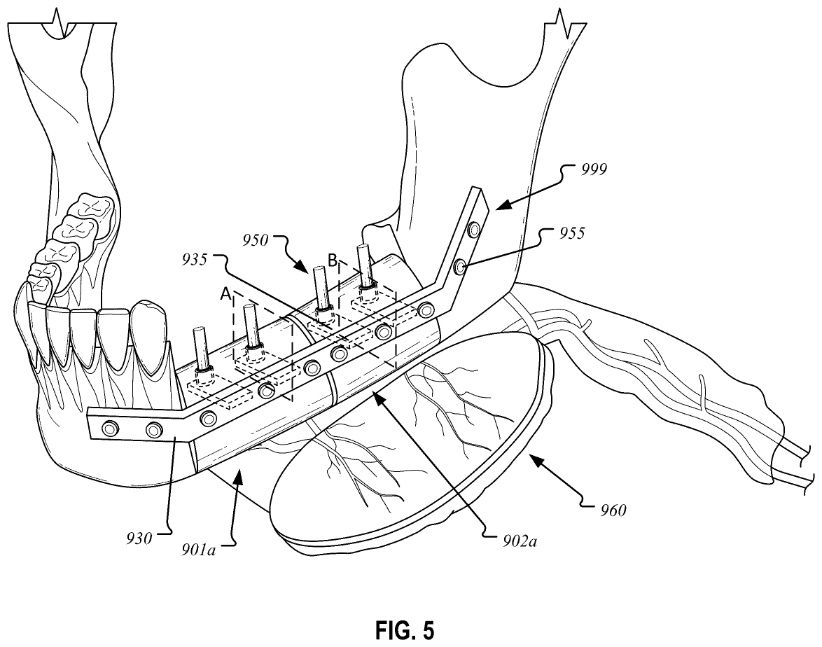

FIG. 5 shows a schematic of a reconstructed mandible, according to an embodiment of the present disclosure. In an embodiment, the reconstruction plate 999 can be fixated to the flanking mandible stumps via multiple of the fastener 955 disposed at various locations along the main bracket 930 of the reconstruction plate 999. The first bone segment 901a and the second bone segment 902a shown can be attached to the plantar plate 935 of the reconstruction plate 999 and secured to the reconstruction plate 999 also via multiple of the fastener 955 disposed at various locations along the main bracket 930 of the reconstruction plate 999 corresponding to the locations of the bone attachment opening 910a, and the dental implant 950 can be inserted through the dental implant opening 915a and coupled to the aperture 940 of the plantar plate 935. Also shown are arteries 960 from the fibula free flap that can be re-attached or reconnected to existing nearby arteries to provide a blood supply and vasculitis-free flap.

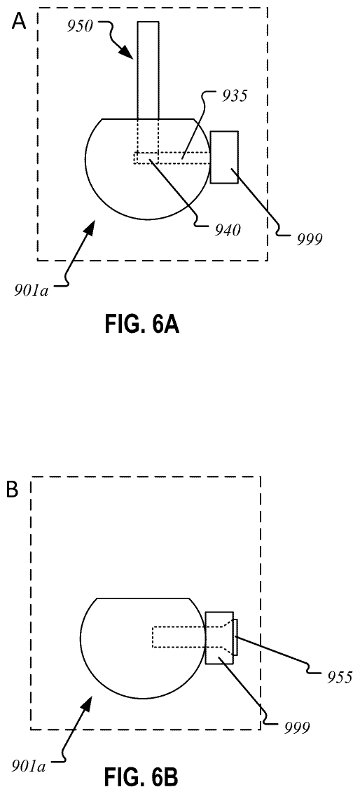

FIG. 6A shows a cross-sectional schematic of plane A, according to an embodiment of the present disclosure. In an embodiment, the cross-sectional plane A shows an orthogonal view to the axis of the bone along where the dental implant 950 is coupled to the plantar plate 935 through the first bone segment 901a via the dental implant opening 915a. Also of note, the top portion of the first bone segment 901a is shown as being planarized instead of rounded. This planarization can be performed as desired based on the dental implant 950 used and eventual dental prostheses installed.

FIG. 6B shows a cross-sectional schematic of plane B, according to an embodiment of the present disclosure. In an embodiment, the cross-sectional plane B shows an orthogonal view to the axis of the bone along where the fastener 955, which is shown as a bone screw, has been screwed through the reconstruction plate 999 and into the first bone segment 901a to secure the main bracket 930 to the first bone segment 901a.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Embodiments of the present disclosure may also be as set forth in the following parentheticals.
(1) A mandible reconstruction and dental implant system, including a first reconstruction template, including a body, a first cutting guide disposed at a first edge of the body and a second cutting guide disposed at a second edge of the body, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template; and a reconstruction plate, including a main bracket, and a first plantar plate protruding orthogonally from the main bracket, the first plantar plate including an aperture.
(2) The system of (1), wherein the main bracket includes at least one opening configured to receive a fastener therein to attach the main bracket to an abutting bone segment.
(3) The system of either (1) or (2), wherein the aperture includes threading for receiving and frictionally securing a complementary threading inserted therein.
(4) The system of any one of (1) to (3), further comprising a positioning pin configured to be inserted through an opening in a bone formed using the dental implant opening and coupled to the plantar plate.
(5) The system of any one of (1) to (4), further comprising a dental implant configured to be inserted through an opening in a bone formed using the dental implant opening and coupled to the plantar plate.
(6) The system of any one of (1) to (5), wherein an angle of a plane of the first cutting guide is based on an obtained medical image.
(7) The system of any one of (1) to (6), wherein an angle of a plane of the second cutting guide is based on the obtained medical image.
(8) The system of any one of (1) to (7), wherein a cross-sectional shape of the plantar plate slot template is rectangular, and a cross-sectional shape of the plantar plate is a complementary rectangular shape to the plantar plate slot template.
(9) The system of any one of (1) to (8), wherein a cross-sectional shape of the bone template is C-shaped and the bone template is configured to engage a donor bone via a push fit.
(10) The system of any one of (1) to (9), wherein a cross-sectional shape of the bone template is circular with split semicircles, and the bone template is configured to engage a donor bone by opening the bone template and arranging the bone template on the donor bone.
(11) The system of any one of (1) to (10), further comprising a second reconstruction template, the first reconstruction template and the second reconstruction template being attached to one another via a template segment connector.
(12) The system of any one of (1) to (11), wherein an arrangement of the plantar plate slot template is aligned with the bone fastener template along a length of the body.
(13) The system of any one of (1) to (12), wherein the arrangement of the bone fastener template is not aligned with the implant template along the length of the body.
(14) The system of any one of (1) to (13), wherein the reconstruction plate includes a second plantar plate slot disposed proximal to the first plantar plate slot and the reconstruction template includes a second plantar plate slot template arranged at a corresponding relative location as the first plantar plate slot to the second plantar plate slot.
(15) The system of any one of (1) to (14), wherein a material of the reconstruction plate is titanium and the reconstruction plate is formed via additive manufacturing.
(16) The system of any one of (1) to (15), wherein a first side of the main bracket of the reconstruction template is configured to be fixated to a first mandible stump and a second side of the main bracket of the reconstruction template is configured to be fixated to a second mandible stump.
(17) A reconstruction template apparatus, including a main bracket; and a first plantar plate protruding orthogonally from the main bracket, the first plantar plate including an aperture.
(18) The apparatus of (17), wherein the main bracket includes at least one opening configured to receive a fastener therein to attach the main bracket to an abutting bone segment.
(19) The apparatus of either (17) or (18), wherein the aperture includes threading for receiving and frictionally securing a complementary threading inserted therein.
(20) The apparatus of any one of (17) to (19), wherein a first side of the main bracket of the reconstruction template is configured to be fixated to a first mandible stump and a second side of the main bracket of the reconstruction template is configured to be fixated to a second mandible stump.
(21) A method of reconstructing a mandible, including obtaining a dental image of the mandible including a degraded portion for resection; attaching a reconstruction template to a donor bone, the reconstruction template including a body, a first cutting guide disposed at a first edge of the body and a second cutting guide disposed at a second edge of the body, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template; forming openings in the donor bone using the first plantar plate slot template, the first bone fastener template, and the first implant template; cutting the donor bone along the first cutting guide and the second cutting guide to generate a first bone segment; attaching a reconstruction plate to the first bone segment via the opening corresponding to the first plantar plate slot template, the reconstruction plate including a main bracket and a first plantar plate protruding orthogonally from the main bracket, the first plantar plate including an aperture; attaching the reconstruction plate with the attached first bone segment to the mandible; and attaching a dental implant to the first plantar plate through the attached first bone segment via the opening formed corresponding to the implant template.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A mandible reconstruction and dental implant system, comprising:
a first reconstruction template, including
a body,
a first cutting guide disposed at a first edge of the body and a second cutting guide disposed at a second edge of the body,
a first plantar plate slot template disposed along the body, the first plantar plate slot template being an opening configured to receive a drill therein,
a first bone fastener template disposed along the body, the first bone fastener template being an opening configured to receive the drill therein, and
a first implant template disposed along the body and aligned with the first plantar plate slot template, the first implant template being an opening configured to receive a drill therein, an axis through the first implant template being orthogonal to an axis through the first plantar plate slot template; and
a reconstruction plate, including
a main bracket, and
a first plantar plate protruding orthogonally from the main bracket, the first plantar plate including an aperture.

2. The system of claim 1, wherein the main bracket includes at least one opening configured to receive a fastener therein to attach the main bracket to an abutting bone segment.

3. The system of claim 1, wherein the aperture includes threading for receiving and frictionally securing a complementary threading inserted therein.

4. The system of claim 1, further comprising a positioning pin configured to be inserted through an opening in a bone formed using the dental implant opening and coupled to the plantar plate.

5. The system of claim 1, further comprising a dental implant configured to be inserted through an opening in a bone formed using the dental implant opening and coupled to the plantar plate.

6. The system of claim 1, wherein an angle of a plane of the first cutting guide is based on an obtained medical image.

7. The system of claim 6, wherein an angle of a plane of the second cutting guide is based on the obtained medical image.

8. The system of claim 1, wherein
a cross-sectional shape of the plantar plate slot template is rectangular, and
a cross-sectional shape of the plantar plate is a complementary rectangular shape to the plantar plate slot template.

9. The system of claim 1, wherein a cross-sectional shape of the bone template is C-shaped and the bone template is configured to engage a donor bone via a push fit.

10. The system of claim 1, wherein a cross-sectional shape of the bone template is circular with split semicircles, and the bone template is configured to engage a donor bone by opening the bone template and arranging the bone template on the donor bone.

11. The system of claim 1, further comprising a second reconstruction template, the first reconstruction template and the second reconstruction template being attached to one another via a template segment connector.

12. The system of claim 1, wherein an arrangement of the plantar plate slot template is aligned with the bone fastener template along a length of the body.

13. The system of claim 12, wherein the arrangement of the bone fastener template is not aligned with the implant template along the length of the body.

14. The system of claim 1, wherein the reconstruction plate includes a second plantar plate slot disposed proximal to the first plantar plate slot and the reconstruction template includes a second plantar plate slot template arranged at a corresponding relative location as the first plantar plate slot to the second plantar plate slot.

15. The system of claim 1, wherein a material of the reconstruction plate is titanium and the reconstruction plate is formed via additive manufacturing.

16. The system of claim 1, wherein a first side of the main bracket of the reconstruction template is configured to be fixated to a first mandible stump and a second side of the main bracket of the reconstruction template is configured to be fixated to a second mandible stump.

17. A reconstruction template apparatus, comprising:
a main bracket; and
a first plantar plate protruding orthogonally from the main bracket, the first plantar plate including an aperture.

18. The apparatus of claim 17, wherein the main bracket includes at least one opening configured to receive a fastener therein to attach the main bracket to an abutting bone segment.

19. The apparatus of claim 17, wherein the aperture includes threading for receiving and frictionally securing a complementary threading inserted therein.

20. The apparatus of claim 17, wherein a first side of the main bracket of the reconstruction template is configured to be fixated to a first mandible stump and a second side of the main bracket of the reconstruction template is configured to be fixated to a second mandible stump.

21. A method of reconstructing a mandible, comprising:
obtaining a dental image of the mandible including a degraded portion for resection;
attaching a reconstruction template to a donor bone, the reconstruction template including a body, a first cutting guide disposed at a first edge of the body and a second cutting guide disposed at a second edge of the body, a first plantar plate slot template disposed along the body, the first plantar plate slot template being an opening configured to receive a drill therein, a first bone fastener template disposed along the body, the first bone fastener template being an opening configured to receive the drill therein, and a first implant template disposed along the body and aligned with the first plantar plate slot template, the first implant template being an opening configured to receive a drill therein, an axis through the first implant template being orthogonal to an axis through the first plantar plate slot template;
forming openings in the donor bone using the first plantar plate slot template, the first bone fastener template, and the first implant template;
cutting the donor bone along the first cutting guide and the second cutting guide to generate a first bone segment;
attaching a reconstruction plate to the first bone segment via the opening corresponding to the first plantar plate slot template, the reconstruction plate including a main bracket and a first plantar plate protruding orthogonally from the main bracket, the first plantar plate including an aperture;
attaching the reconstruction plate with the attached first bone segment to the mandible; and
attaching a dental implant to the first plantar plate through the attached first bone segment via the opening formed corresponding to the implant template.
